(19) United States
(12) Patent Application Publication
(10) Pub. No.: US 2016/0287348 A1
(43) Pub. Date: Oct. 6, 2016

(54) MALLEABLE WAVEGUIDE

(71) Applicant: Invuity, Inc., San Francisco, CA (US)

(72) Inventors: Alex Vayser, Mission Viejo, CA (US); Aaron Weiss, Oakland, CA (US)

(21) Appl. No.: 15/080,350

(22) Filed: Mar. 24, 2016

Related U.S. Application Data
(60) Provisional application No. 62/140,332, filed on Mar. 30, 2015.

(51) Int. Cl. A61B 90/30; A61B 1/00; A61B 1/04; A61B 1/07; A61B 1/06

(52) U.S. Cl. CPC A61B 90/30; A61B 1/07; A61B 1/0661; A61B 1/04; A61B 1/00147; A61B 2090/306

(57) ABSTRACT

A method for illuminating a treatment target includes providing a medical device and a malleable waveguide, and positioning the medical device and the malleable waveguide into the treatment target. The method also includes deforming the medical device to conform with a native anatomy in the treatment target, and deforming the malleable waveguide to conform with the native anatomy in the treatment target. The deformation of the malleable waveguide cooperates with the deformation of the medical device. The method also includes performing a medical procedure with the medical device and illuminating the treatment target with light from the malleable waveguide.

### CROSS-REFERENCE TO RELATED APPLICATIONS

The present application is a non-provisional of, and claims the benefit of U.S. Provisional Application No. 62/140,332 (Attorney Docket No. 40556-744.101) filed Mar. 30, 2015; the entire contents of which are incorporated herein by reference

### BACKGROUND OF THE INVENTION

1. Field of the Invention

The present application generally relates to medical devices, systems and methods, and more particularly relates to malleable waveguides that may be used with other medical devices or instruments to illuminate a target.

Current illuminated surgical instruments are most commonly produced with fiber optic bundles. These bundles are often housed in rigid or semi rigid tubing that holds the fibers. Fibers have limitations because the light being extracted out of the distal end is not shaped well, directed or uniform. Therefore the target may not be illuminated effectively. Also, because the fibers are in rigid tubing, the fibers cannot be easily bent to direct light in a desired direction, nor can the fibers easily conform to an instrument that is also malleable and flexed during use. Also, fiber optic bundles are not efficient at transmitting light.

Other waveguide technology may be more robust but the waveguide is often molded and is rigid, such as seen with common optical polymers such as acrylic, polycarbonate, cyclo olefin polymer (COP) or cyclo olefin copolymer (COC). The rigidity of these devices may limit their use. Especially when combined with an instrument that is malleable and thus the waveguide cannot flex with the malleable instrument. This also may create a problem when a readily available waveguide is used with various readily available shaped retractors, especially those that are curved because coupling a rigid waveguide having a pre-formed shape to the curved retractor having a different shape is not easily done unless the two components are designed to mate with one another or supplied coupled together from the manufacturer. Therefore, it would be desirable to provide a malleable waveguide that is easily conformable with the instrument to which it is coupled, and also it would be desirable to provide a malleable waveguide that delivers light efficiently, safely and that can shape and direct the light to a target. At least some of these objectives will be met by the exemplary embodiments described herein.

There are some commercially available surgical instruments which include malleable fiber optics bundles that are attached to the instrument, such as a retractor blade. However, there is still an issue of light shaping and directionality that is not provided by the fiber optics, especially when they are adjacent or attached to surgical instruments. Since light output from the fibers is conical, much of the output is obstructed by the device. It would therefore be desirable to provide malleable or moldable illumination elements such as a waveguide that may be used with an instrument such as a hand held device or retractor, or any other surgical instrument to provide light directionality, thermal stability and shaping of the light.

### SUMMARY OF THE INVENTION

The present invention generally relates to medical systems, devices and methods, and more particularly relates to malleable waveguides that may be used with other medical devices and instruments to illuminate a target.

In a first aspect, a method for illuminating a treatment target comprises providing a medical device and a malleable waveguide, positioning the medical device and the malleable waveguide into the treatment target, and deforming the medical device to conform with a native anatomy in the treatment target. The method also comprises deforming the malleable waveguide to conform with the native anatomy in the treatment target, wherein the deformation of the malleable waveguide cooperates with the deformation of the medical device, performing a medical procedure with the medical device, and illuminating the treatment target with light from the malleable waveguide.

The medical device may comprise a surgical retractor, a suction tube, a suction coagulator, a laparoscopic instrument, an electrosurgical or other energy delivery instrument, or a catheter. The malleable waveguide may be formed primarily of silicone. The malleable waveguide may comprise an index of refraction of 1.40 or higher, may have an optical transmission efficiency of 90% or greater, or may have an operating range of between about −45 degrees Celsius and about 200 degrees Celsius.

The method may further comprise coupling a fiber optic cable to the malleable waveguide, or inputting light from a light source into the malleable waveguide. The method may further comprise imaging the treatment target with an imaging element. The method may further comprise steering the medical device, and the malleable waveguide may steer with the medical device, thereby cooperating with the steering.

The method may further comprise coupling the malleable waveguide with the medical device such that the malleable waveguide conforms to a contour of the medical device. The method may further comprise radially expanding the medical device and radially expanding the malleable waveguide with the medical device. The method may further comprise performing an electrosurgical procedure with the medical device. The malleable waveguide may comprise optical microstructures for extracting light therefrom, and the optical microstructures may direct the extracted light toward the treatment target. The microstructures may shape the extracted light and direct the extracted light toward the treatment target. The malleable waveguide may have a stem. A stem is a portion of the waveguide where no intentional light extraction takes place. Light is simply transferred along its path. The stem is also desirable in any waveguide as it helps light input into a waveguide mix more uniformly and allows the light to bounce at least once off a wall of the waveguide or stem which improves transmission efficiency. Optionally, the method may further comprise illuminating the treatment target with light emitted from an optical fiber disposed adjacent or integrated within in the malleable waveguide.

In another aspect, a system for illuminating a treatment target comprises a deformable medical device, and a malleable waveguide coupled to the medical device. The malleable waveguide conforms to the deformable waveguide upon deformation of the deformable medical device to conform with native anatomy in the treatment target. The malleable waveguide illuminates the treatment target with light emitted therefrom.

The medical device may comprise a surgical retractor, a suction tube, a suction coagulator, a laparoscopic instrument, an electrosurgical or energy instrument, or a catheter. The malleable waveguide may be formed primarily of silicone. The malleable waveguide may comprise an index of refraction of 1.40 or higher, or have an optical transmission efficiency of 90% or greater, or may have an operating range of between about −45 degrees Celsius and about 200 degrees Celsius.

The system may further comprise a fiber optic cable coupled to the malleable waveguide, or may further comprise an external light source optically coupled with the malleable waveguide. The system may further comprise an imaging element coupled with the medical device or the optical waveguide. The medical device may comprise a steering mechanism for controlling a shape of the medical device, and the malleable waveguide may steer with the medical device, thereby cooperating with the steering mechanism.

The malleable waveguide may be coupled with the medical device such that the malleable waveguide conforms to a contour of the medical device. The medical device may have an expanded configuration and a collapsed configuration, and expansion of medical device from the collapsed configuration to the expanded configuration may expand the malleable waveguide. The medical device may be an electrosurgical or energy instrument. The malleable waveguide may comprise optical microstructures for extracting light therefrom, and the optical microstructures may direct the extracted light toward the treatment target. The microstructures may shape the extracted light and direct the extracted light toward the treatment target. An input stem coupled to a proximal portion of the waveguide is also desirable as the stem helps light to mix in the waveguide and also helps the light bounce at least once off a wall along the waveguide or stem improving transmission efficiency. Optionally, the system may further comprise an optical fiber disposed adjacent or integrated within in the malleable waveguide, the optical fiber configured to illuminate the treatment target with light emitted therefrom.

In still another aspect, a malleable surgical illumination element comprises an optical waveguide formed from a malleable polymeric material. The waveguide is bendable in any direction into a desired configuration.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B shows an exemplary embodiment of an illuminated medical instrument.

FIG. 2 illustrates an exemplary embodiment of an illuminated steerable medical instrument.

FIGS. 3A-3B illustrate an exemplary embodiment of a malleable waveguide coupled to a bendable medical instrument.

FIGS. 4A-4B illustrate an exemplary embodiment of an illuminated and expandable frame.

FIG. 5 illustrates an exemplary embodiment of an illuminated surgical retractor.

FIGS. 6A-6C illustrate an exemplary embodiment of a malleable waveguide.

FIG. 7 illustrates an exemplary embodiment of an illuminated electrosurgical instrument.

FIGS. 8A-8C illustrate a bendable waveguide.

FIGS. 9A-9B illustrate an actuatable instrument.

FIG. 10 illustrates an exemplary embodiment of a hybrid malleable waveguide.

### DETAILED DESCRIPTION OF THE INVENTION

Specific embodiments of the disclosed device, delivery system, and method will now be described with reference to the drawings. Nothing in this detailed description is intended to imply that any particular component, feature, or step is essential to the invention.

Polydimethylsiloxane (commonly referred to as silicone) is a promising optical material with desired mechanical properties. It may have transmission better than 90% over a 2 mm optical path length which means light may be transmitted from a light source to a target efficiently without drastic losses. In still other embodiments, transmission may be 50% or higher along a 2 mm optical path. Any efficiency may apply to any of the embodiments described herein. Additionally, silicone elastomers may be used for optical components and have a working temperature range between −45 degrees Celsius and 200 degrees Celsius, with an index of refraction over 1.40. In other embodiments, the index of refraction may be 1.33 or greater, for example in a liquid light guide, or the index of refraction may be 1.0 or greater for a hollow waveguide with an air gap, or the index of refraction could be 1.4 or higher for other polymers. Any of these indices of refraction may apply to any of the waveguides disclosed herein. A typical manufacturer of this material includes Dow Corning which produces moldable silicone 1000 and 2000 series.

The durometer of the silicone material may be soft enough to provide a malleable waveguide which can be injection molded or otherwise formed using techniques known in the art. The molded waveguide can then be attached to a fiber bundle or integrated into a fiber optic bundle such as a pigtail connection. The malleable waveguide can also be directly coupled to a light source such as a light emitting diode (LED). Thus, any light source may be used to provide light to the waveguide. Microstructures or other optical surface features may be formed into the malleable waveguide that are used to extract and direct the light onto a target, such as a surgical target. Additionally, various optical coatings and claddings may be applied to the waveguide or in between the waveguide and an instrument in order to provide desirable optical properties. Air gaps may also be disposed between the waveguide and an adjacent instrument in order to minimize light leakage from the waveguide.

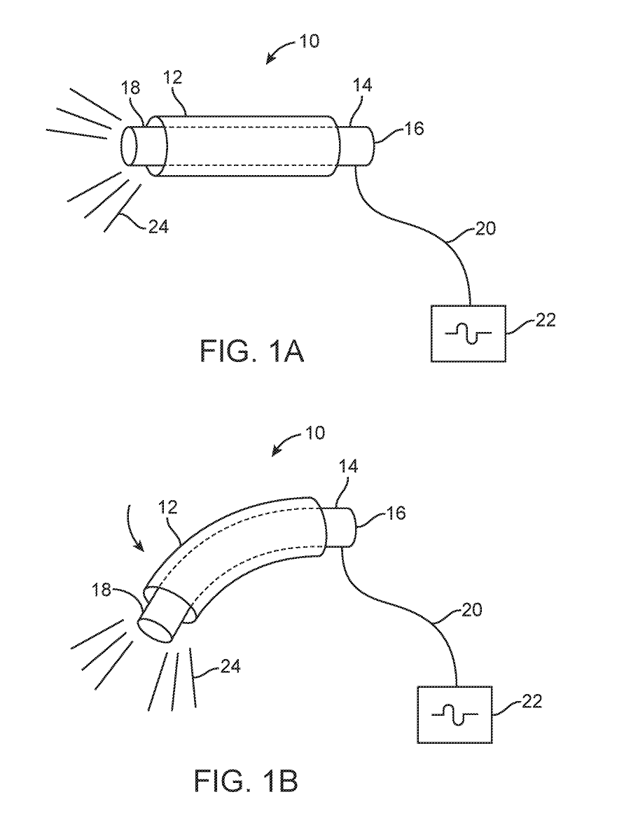

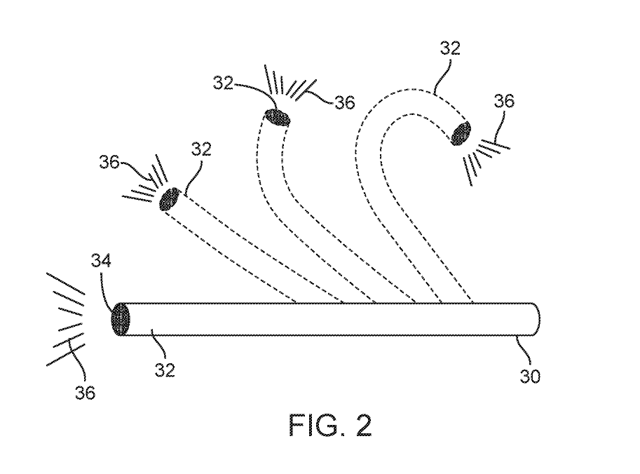

FIGS. 1A-1B illustrate a medical device 10 having a malleable waveguide 12 coupled to a surgical instrument such as a suction tube 14. The suction tube 14 is preferably a malleable metal such as stainless steel and has a proximal end 16 and a distal end 18. The proximal end may be coupled to tubing which allows a vacuum source to be coupled to the suction tube so that the distal end 18 can remove fluids or other debris from a surgical field. A malleable waveguide 12 is either molded over the suction tube 12, or may be molded separately and then fit over the suction tube. A proximal end of the waveguide may be coupled to a light source such as an LED, or an external light source such as a xenon light box via a fiber optic cable (not illustrated). Light therefore is input into the proximal part of the waveguide and the light travels through the waveguide and the light 24 exits the distal portion of the waveguide to illuminated the surgical target with the desired intensity, spot size, or other optical properties so that the surgeon can see the surgical target. Optionally, the suction tube may also be a suction coagulator and thus the metal suction tube may also be a conductor for conducting energy from an external power source 22 via a power cable 20. The distal portion of the suction tube may contact target tissue or have electrodes that contact the target tissue, and deliver the energy to the target tissue. The surgeon may bend the suction tube as seen in FIG. 1B in order to conform with the local anatomy in order to suction a desired area of the target. Therefore, the malleable waveguide also bends with the suction tube so that the light exits the waveguide and illuminates the target where the suctioning is being performed. In this embodiment or any embodiment, the malleable material may be plastically deformed and retain the bent shape, or it may be resilient and spring back to its unbiased shape unless constrained and held in its bent position by the deformed suction tube or instrument. The malleable waveguide may be formed from any malleable material with desired optical and mechanical properties, but preferred embodiments are formed from any of the silicones disclosed herein. In other embodiments, the suction tube may be replaced with a solid rod or any other instrument, including for example a laparoscopic grasper.

In other embodiments, the malleable waveguide may be coupled to any other instrument such as a surgical instrument. Exemplary surgical instruments include but are not limited to catheters, laparoscopies instruments, robotically controlled instruments including catheter shafts and laparoscopic instruments. The malleable waveguide may be slidably disposed over the instrument like a glove disposed over a hand, or the malleable waveguide may be fixedly or releasably coupled to the instrument using known mechanical or other attachment methods.

FIG. 2 illustrates still another exemplary embodiment where a malleable waveguide may be combined with a flexible and steerable medical device. Here, flexible catheter 30 may include a malleable waveguide 32 at its tip 34 so that light 36 exiting the catheter exits distally from the tip of the catheter. FIG. 2 shows the catheter bending or being steered into different configurations (shown in phantom) and the waveguide bends with the catheter therefore the light continues to exit distally from the catheter tip. An optional imaging element (not shown) may also be included in the tip of the catheter and thus the tip may be bent into a desired direction to capture an image with the imaging element in that desired direction, and light from the waveguide will illuminate the target so that the imaging element can capture an image of the target. The catheter shape may be controlled by one or more pull wires in running through the catheter shaft and which are actuated by a handle (not shown) coupled to the proximal portion of the catheter shaft. The light may be provided from LEDs in the handle, or from an external light source coupled to the waveguide with fiber optics. Instruments, imaging elements, or other sensors may be passed through the catheter to a target treatment site. The catheter may be an endoscope, except instead of using traditional optical fibers for illumination, a malleable waveguide may be used to conduct light.

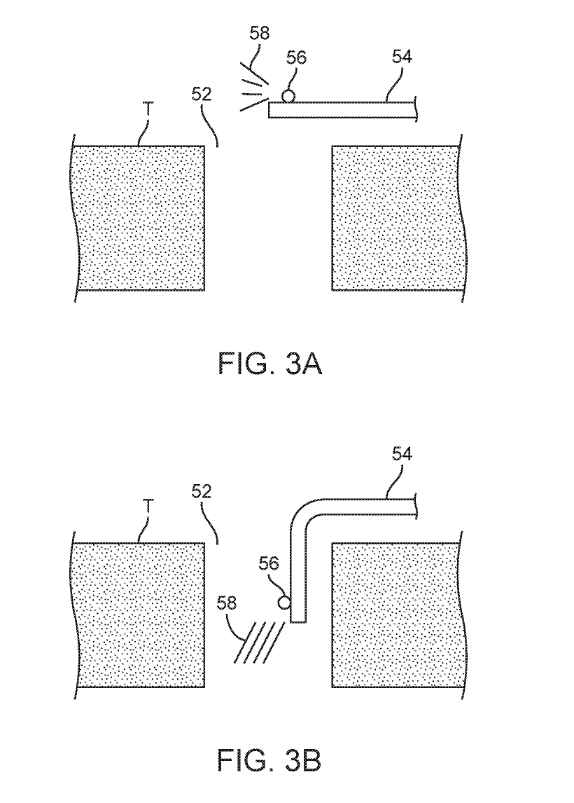

FIGS. 3A and 3B illustrate another exemplary embodiment where a malleable waveguide is desirable. In FIG. 3A an incision 52 has been made in tissue T in a patient to provide access to a surgical target. A flat retractor blade 54 having an illumination element 56 emitting light 58 may be used in the surgical procedure to retract tissue to open the incision and provide maximum access to the surgical target. In the case of a retractor blade, here a flat blade that does not conform to the anatomy, often times a surgeon will bend the retractor blade to conform to the anatomy, as seen in FIG. 3B where the blade has been bent approximately ninety degrees so that the tip of the blade enters into the incision and then the blade can be used to illuminate the surgical target with light 58 and so that the blade can be used to retract the tissue. Rigid illumination elements do not always bend with the retractor blade, therefore combining a malleable waveguide with the retractor blade allows the waveguide to bend with the retractor blade and conform to the anatomy and the light is directed toward the target. The waveguide may be formed form any malleable material with the desired optical and mechanical properties, including any of the silicones disclosed herein. Other retractors may be curved or otherwise contoured in shape and therefore it is also advantageous if the waveguide conforms with the contours and curves of the retractor blade. The waveguide may be molded over the retractor blade or fitted together and integrated into an assembly. Attaching the waveguide to the retractor blade, or any instrument, provides a lower profile instrument which is desirable so that the assembly does not occupy too much space in the surgical field. An exemplary retractor may be a malleable spatula.

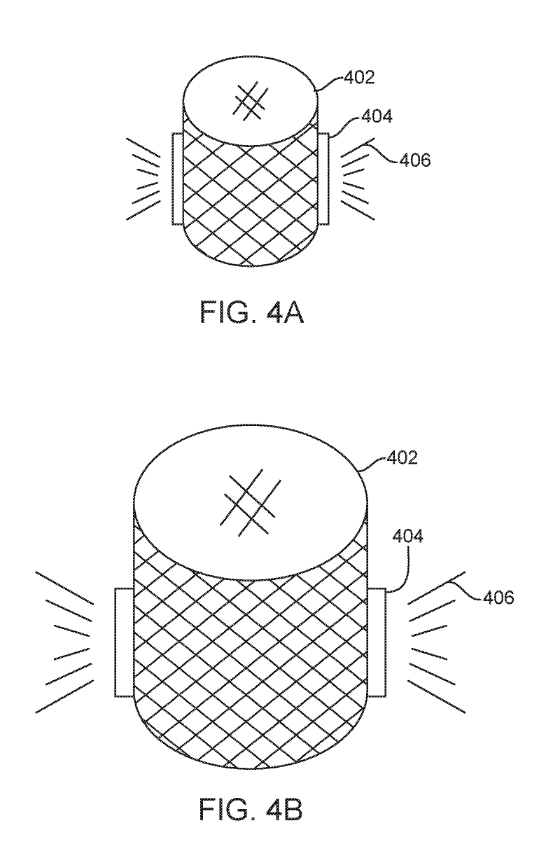

FIGS. 4A-4B illustrate another exemplary embodiment where a malleable waveguide may be coupled to an expandable device, such as a stent or other expandable frame. The frame 402 may be a radially expandable frame that either self-expands using self-expanding materials such as nitinol, or that can be expanded over a device such as a balloon to plastically deform the frame. A malleable waveguide 404 may be coupled to the frame and the waveguide expands with the frame from the collapsed configuration seen in FIG. 4A to the expanded configuration in FIG. 4B and light 406 may be emitted from the waveguide. An exemplary frame includes self-expanding stent or a balloon expandable stent, both of which are well known in the art. The waveguide may be any of the waveguides or materials disclosed herein, including any of the malleable silicone materials. The expandable frame may be inserted into an incision to illuminate the surgical field. The frame opens to keep the shape and conform to the incision, or the frame can be designed to over expand thereby retracting the tissue. In the embodiment of FIGS. 4A-4B, the light is seen exiting radially outward away from the frame, but the light may also exit radially inward toward the central axis or any desired location. A local light source such as an LED may be coupled to the waveguide, or an external light source may provide light to the waveguide such as via a fiber optic cable.

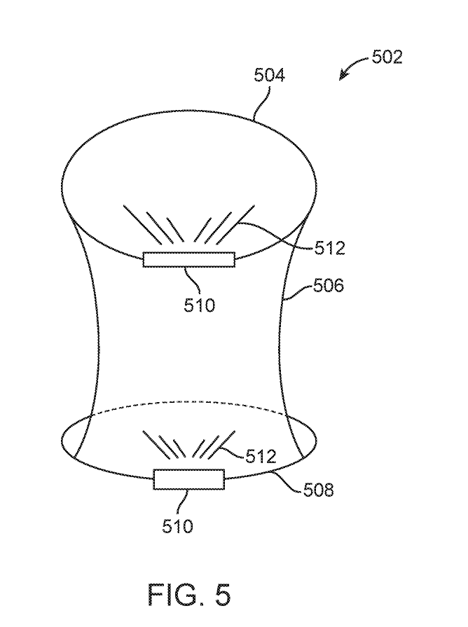

FIG. 5 illustrates another exemplary embodiment of a retractor device 502 having a malleable waveguide 510 coupled to the device to provide light 512 to the surgical field. The retractor device 502 includes an upper ring 504 and a lower ring 508. A flexible membrane 506 couples the two rings together. The lower ring 508 is usually placed in the surgical wound and the upper ring 504 remains outside the wound. When the upper ring 504 is rotated around itself, the membrane 506 spools around the ring 504 and tensions the membrane 506 thereby retracting tissue in the wound. The malleable waveguide which may be integrated into the top or bottom or both rings 504, 508 provides lighting to the surgical field and the waveguide will conform to the shape of the rings as they are collapsed to be positioned in the wound, or as they are rotated to tension the membrane. Retractors of this type are known in the art, such as those manufactured by Applied Medical. The flexible membrane may be a light conducting material such as silicone and may also serve as the waveguide and light may be input into the membrane and the flexible may deliver the light to a target.

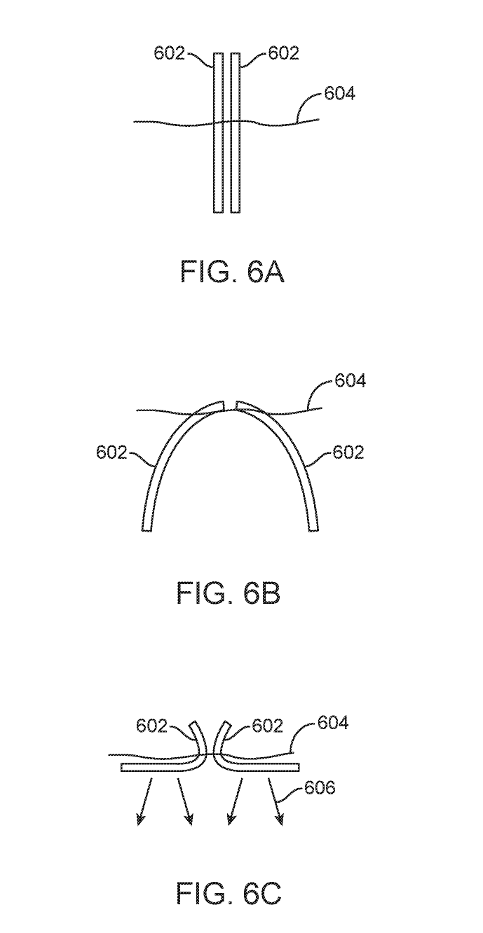

FIGS. 6A-6C illustrate still another exemplary embodiment if a malleable waveguide 602. Outside of laparoscopic instruments mentioned earlier, there is also potential to develop malleable devices 602 that open up in the incision. For example, in FIG. 6A, a pair of straight flat waveguides 602 are inserted into the incision. Once inserted, in FIG. 6B each of the waveguides expands and attaches to the peritoneum 604 for example to provide overhead illumination 606 of the cavity. The malleable waveguide 602 can open up and contour to the shape of the surface as seen in FIG. 6C. Magnets for example may be used to keep the waveguide attached to the surface. Magnets may be placed on the surface of the skin and pull the waveguide towards it. These waveguides may contain imaging elements as well. In alternative embodiments, wires may pass through the waveguide and actuation of the wires control the shape of the waveguide as disclosed in greater detail below.

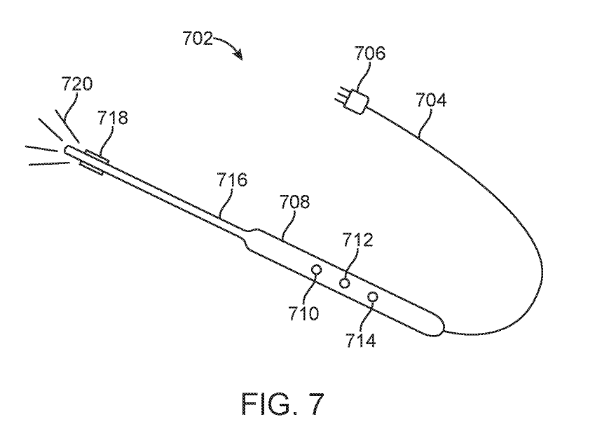

FIG. 7 illustrates another exemplary embodiment of a surgical instrument 702 coupled to a malleable waveguide 718. Here the surgical instrument 702 is an electrosurgical pencil having a handle 708 with an electrode 716 for delivering current to tissue. A power cord 704 having a coupling 706 allows the device to be operatively coupled to an external power source such as an RF generator. Controls 701, 712, 714 may allow the operator to deliver energy to the electrode 716 that preferably cut, preferably coagulate, or illuminate the target. The malleable waveguide 718 may be coupled to the tip of the electrode to illuminate 720 the surgical field. The electrode may be bent to conform to the anatomy and thus the waveguide will also bend with the electrode. Light may be input into the waveguide by coupling a proximal portion of the waveguide with an external light source via fiber optic cable, or LEDs or other illumination elements may input light into the proximal portion of the waveguide. In this embodiment the waveguide is illustrated as being a discrete element disposed along a distal portion of the electrode 716. In other embodiments, the waveguide may be a layer of material disposed along the entire length of the electrode and therefore the light may be emitted from the electrode and the light source (e.g. LEDs) may be disposed in the handle or on the distal tip of the handle. The malleable waveguide will conform to the electrode as the electrode is manipulated by an operator such as a surgeon in order to conform to the anatomy.

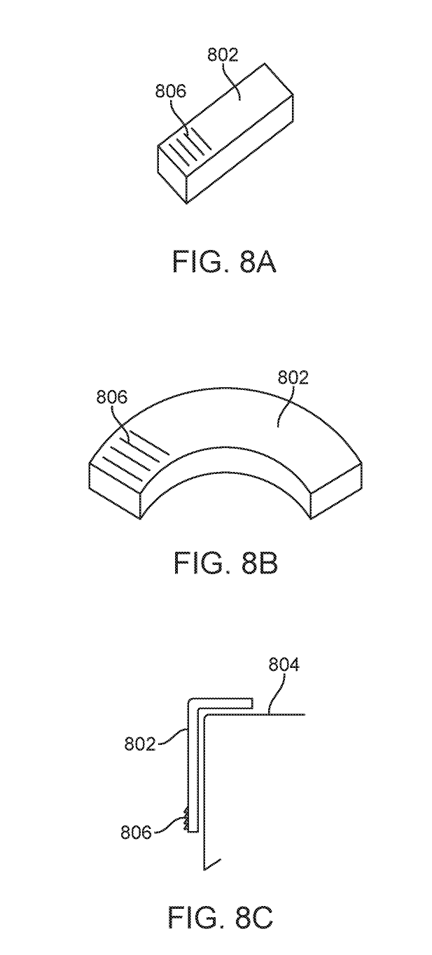

FIGS. 8A-8B illustrate a malleable waveguide 802 that may be bent in any direction as required. FIG. 8A shows the waveguide 802 in a generally flat and planar configuration, while in FIG. 8B the waveguide 802 has been bent into an arcuate configuration. The waveguide may be made from any malleable optical material such as silicone and bent into any desired configuration in order to fit into the work space or to be mated with another instrument. Light may be input into the waveguide with fiber optic cables which are either releasably coupled to the waveguide or fixedly coupled to the waveguide. In still other embodiments, illumination elements adjacent the waveguide may be used to input light into the waveguide, such as an LED or an array of LEDs. Any of the optical structures 806 described herein such as microstructures, lenslets, prisms, etc. may be disposed on the waveguide to help extract and direct the extracted light toward a target. FIG. 8C illustrates the waveguide 802 bent into an L, or approximately 90 degree angle in order to conform with the shape of a surgical retractor 804. The waveguide may be bent into any shape in order to conform with the contours or shape of any retractor. As previously mentioned, the waveguide may be plastically deformed to retain the deformed shape, or it may be resilient and return to its unbiased shape.

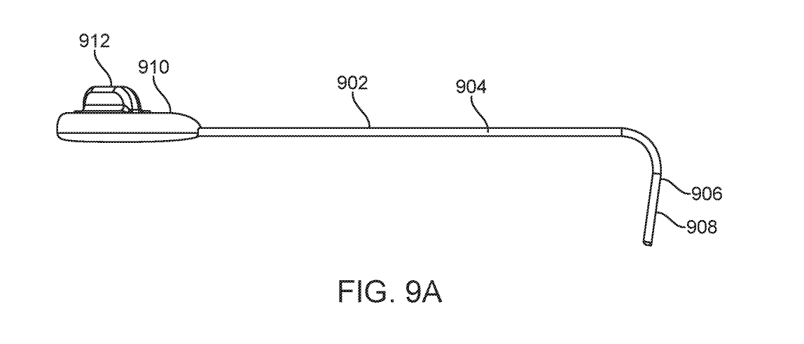

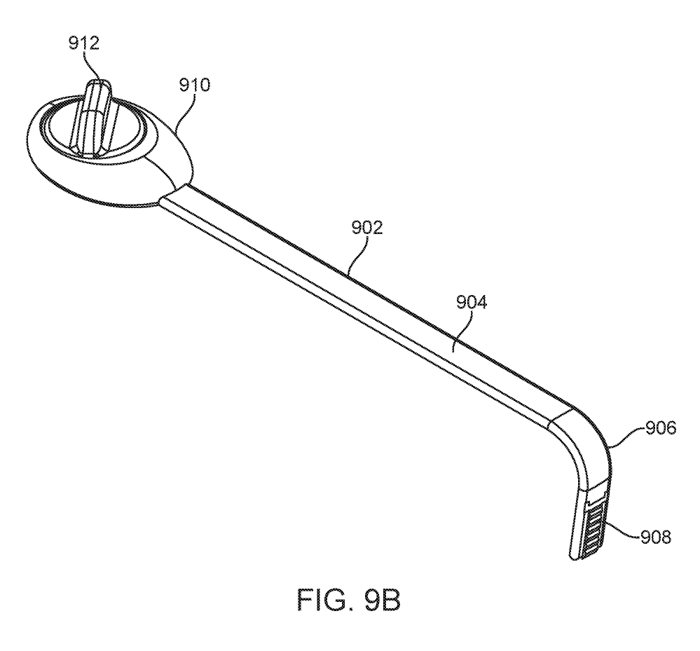

FIGS. 9A-9B illustrate an actuatable instrument 902 which includes an elongate shaft 904 having one or more actuatable pull wires (not illustrated) disposed in the shaft. A distal end of the one or more pull wires is coupled to a distal portion of the actuatable shaft 904 and a proximal end of the one or more pull wires is coupled to an actuation mechanism 912 disposed on a handle 910 coupled to the shaft. Actuation of the actuation mechanism increases or decreases tension in the wires thereby bending the distal portion of the shaft into a desired configuration. The actuation mechanism may be any mechanism to control movement of the pull wires. Here, the mechanism is a rotatable knob. For example, in FIG. 9A, the tip is bent into an L-shape that is approximately ninety degrees. Depending on the number of pull wires, the tip may be bent into any desired configuration in one, two or three dimensions. A waveguide 908 such as any of those described herein may be coupled to the distal portion of the shaft, thereby allowing the waveguide position to be adjusted as required. FIG. 9B illustrates the features of actuatable instrument 902 in greater detail.

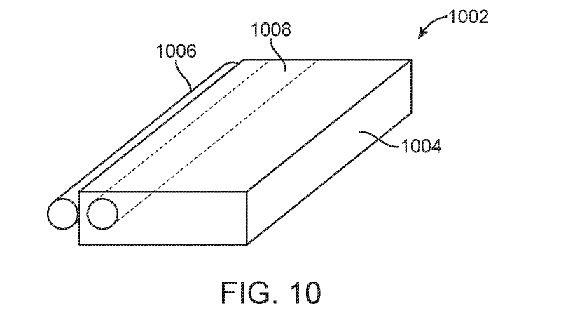

FIG. 10 illustrates another exemplary embodiment of a malleable waveguide 1002. The malleable waveguide 1002 includes a malleable waveguide 1004 and a malleable optical fiber 1006, 1008. One or more optical fibers 1006, 1008 may be coupled to the waveguide as shown by element 1006, or the optical fiber may be integral with and disposed in the waveguide as shown by the optical fiber 1008. This hybrid embodiment provides one index of refraction for the waveguide, and a second index of refraction for the optical fiber that may be the same or different than the waveguide index of refraction. Preferably both the fiber and the waveguide are both malleable so they can bend and conform with the instrument to which they are attached. Light may be supplied by one or more light sources such as an adjacent LED or an external light source such as a xenon light source that is optically coupled to the waveguide and/or optical fiber with a fiber optic cable. In an alternative embodiment, the malleable waveguide includes a hollow channel disposed therethrough. The hollow channel may be in addition to the optical fiber 1006, 1008 or it may be by itself.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for illuminating a treatment target, said method comprising:
providing a medical device and a malleable waveguide;
positioning the medical device and the malleable waveguide into the treatment target;
deforming the medical device to conform with a native anatomy in the treatment target;
deforming the malleable waveguide to conform with the native anatomy in the treatment target, wherein the deformation of the malleable waveguide cooperates with the deformation of the medical device;
performing a medical procedure with the medical device; and
illuminating the treatment target with light from the malleable waveguide.

2. The method of claim 1, wherein the medical device comprises a surgical retractor, a suction tube, a suction coagulator, a laparoscopic instrument, an electrosurgical instrument, or a catheter.

3. The method of claim 1, wherein the malleable waveguide is formed primarily of silicone.

4. The method of claim 1, wherein the malleable waveguide comprises an index of refraction of 1.40 or higher.

5. The method of claim 1, wherein the malleable waveguide comprises an optical transmission efficiency of 90% or greater.

6. The method of claim 1, wherein the malleable waveguide has an operating range of between about −45 degrees Celsius and about 200 degrees Celsius.

7. The method of claim 1, further comprising coupling a fiber optic cable to the malleable waveguide.

8. The method of claim 1, further comprising inputting light from a light source into the malleable waveguide.

9. The method of claim 1, further comprising imaging the treatment target with an imaging element.

10. The method of claim 1, further comprising steering the medical device, and wherein the malleable waveguide steers with the medical device, thereby cooperating with the steering.

11. The method of claim 1, further comprising coupling the malleable waveguide with the medical device such that the malleable waveguide conforms to a contour of the medical device.

12. The method of claim 1, further comprising radially expanding the medical device and radially expanding the malleable waveguide with the medical device.

13. The method of claim 1, further comprising performing an electrosurgical procedure with the medical device.

14. The method of claim 1, wherein the malleable waveguide comprises optical microstructures for extracting light therefrom, and wherein the optical microstructures direct the extracted light toward the treatment target.

15. The method of claim 14, wherein the microstructures shape the extracted light and direct the extracted light toward the treatment target.

16. The method of claim 1, further comprising illuminating the treatment target with light emitted from an optical fiber disposed within or adjacent the malleable waveguide.

17. A system for illuminating a treatment target, said system comprising:
a deformable medical device; and
a malleable waveguide coupled to the medical device, wherein the malleable waveguide conforms to the deformable waveguide upon deformation of the deformable medical device to conform with native anatomy in the treatment target, and wherein the malleable waveguide illuminates the treatment target with light emitted therefrom.

18. The system of claim 17, wherein the medical device comprises a surgical retractor, a suction tube, a suction coagulator, a laparoscopic instrument, an electrosurgical instrument, or a catheter.

19. The system of claim 17, wherein the malleable waveguide is formed primarily of silicone.

20. The system of claim 17, wherein the malleable waveguide comprises an index of refraction of 1.40 or higher.

21. The system of claim 17, wherein the malleable waveguide comprises an optical transmission efficiency of 90% or greater.

22. The system of claim 17, wherein the malleable waveguide has an operating range of between about −45 degrees Celsius and about 200 degrees Celsius.

23. The system of claim 17, further comprising a fiber optic cable coupled to the malleable waveguide.

24. The system of claim 17, further comprising an external light source optically coupled with the malleable waveguide.

25. The system of claim 17, further comprising an imaging element coupled with the medical device or the optical waveguide.

26. The system of claim 17, wherein the medical device comprises a steering mechanism for controlling a shape of the medical device, and wherein the malleable waveguide steers with the medical device, thereby cooperating with the steering mechanism.

27. The system of claim 17, wherein the malleable waveguide is coupled with the medical device such that the malleable waveguide conforms to a contour of the medical device.

28. The system of claim 17, wherein the medical device has an expanded configuration and a collapsed configuration, and wherein expansion of medical device from the collapsed configuration to the expanded configuration expands the malleable waveguide.

29. The system of claim 17, wherein the medical device is an electrosurgical instrument.

30. The system of claim 17, the wherein the malleable waveguide comprises optical microstructures for extracting light therefrom, and wherein the optical microstructures direct the extracted light toward the treatment target.

31. The system of claim 30, wherein the microstructures shape the extracted light and direct the extracted light toward the treatment target.

32. The system of claim 17, further comprising an optical fiber disposed within or adjacent the malleable waveguide, the optical fiber configured to illuminate the treatment target with light emitted therefrom.

33. A malleable surgical illumination element, said element comprising:
an optical waveguide formed from a malleable polymeric material, wherein the waveguide is bendable in any direction into a desired configuration.
